(19) United States
(12) Patent Application Publication
(10) Pub. No.: US 2012/0308497 A1
(43) Pub. Date: Dec. 6, 2012

(54) NAIL POLISH WITH A CRACK AND COLOR CHANGING EFFECT

(76) Inventor: YAO-CHUNG LEE, Kaohsiung City (TW)

(21) Appl. No.: 13/118,599

(22) Filed: May 31, 2011

(51) Int. Cl. A61K 8/73; A61Q 3/02; A61K 8/85

(52) U.S. Cl. 424/61

(57) ABSTRACT

A nail polish with a crack and color changing effect includes a polymer, a solvent, a plasticizer, a pigment, a silica and a thermochromic powder. The polymer accounts for 10%-30% of the total weight and contains nitrocellulose and resin. The solvent accounts for 44%-64% of the total weight. The plasticizer accounts for 3%-8% of the total weight. The silica accounts for 8% of the total weight. The pigment accounts for 10%-20% of the total weight. The thermochromic powder accounts for 5% of the total weight. The nail polish with a crack and color changing effect composed by the aforementioned components may generate cracks after being applied and dried and also generate a color changing effect with the changing of the temperature of nails.

### BACKGROUND OF THE INVENTION

(a) Technical Field of the Invention

The present invention relates to a nail polish, and more particularly relates to a nail polish with a crack and color changing effect.

(b) Description of the Prior Art

In recent years, the sales and marketing of the nail polish products have been increasingly expanding. The fingernail care and nail protection treatment are prevailing on the street stalls, and even the nail painting and a variety of nail care treatments become a popular fashion.

However, the nail polish in the prior art has the following defects in use.

1. The nail polish in the prior art presents a uniform appearance after being applied uniformly and dried. If a different pattern is wanted, the pattern must be drawn by hand and we even need to resort to a nail specialist, which is rather time-consuming.

2. The nail polish after being applied and dried presents the preset color without changes, which is monotonous.

### SUMMARY OF THE INVENTION

In view of the nail polish which presents a smooth and uniform visual effect in the prior art, the inventor of the present invention proposes a nail polish with a crack and color changing effect to overcome the defects and shortcomings of the prior art.

The present invention is directed to providing a nail polish with a crack and color changing effect, which forms a nail polish coating after being applied on nails and generates cracks in the course of drying. The nail polish coating generates a color changing effect due to the changing of temperature of the nails, thus giving a special visual effect of the nails.

To achieve the aforementioned purpose, the nail polish with a crack and color changing effect of the present invention includes:

a polymer, accounting for 10%-30% of the total weight and containing a nitrocellulose and a resin;

a solvent, accounting for 44%-64% of the total weight;

a plasticizer, accounting for 3%-8% of the total weight;

a pigment, accounting for 10%-20% of the total weight;

a silica, accounting for 8% of the total weight; and

a thermochromic powder, accounting for 5% of the total weight.

By the aforementioned technical means, the nail polish with a crack and color changing effect of the present invention may generate a variety of crack effects and further generate a color changing effect at a specific temperature, which greatly improves the applicability of the nail polish.

The resin of the polymer may be at least one selected from a group consisting of: phthalic anhydride, trimellitic anhydride, glycol copolymer and adipic acid, fumaric acid, phthalic acid, tricyclodecane dimethanol copolymer and a mixture thereof.

The solvent contains butyl acetate, ethyl acetate and isopropyl alcohol.

The plasticizer is acetyl tributyl citrate (ATBC).

The nitrocellulose and resin respectively account for 8-10% and 90-92% of the total weight of the polymer.

The thermochromic powder is in a colored state below a specific temperature and turns into a colorless state when the temperature rises to the specific temperature.

### BRIEF DESCRIPTION OF THE DRAWINGS

FIG. 1 is a schematic perspective view illustrating an appearance and operations of the present invention;

FIG. 2 is a schematic perspective view illustrating an appearance and operations of the present invention;

FIG. 3 is a schematic perspective view illustrating an appearance and operations of the present invention, in which the nail polish generates cracks; and

FIG. 4 is a schematic perspective view illustrating an appearance and operations of the present invention, in which the nail polish changes colors.

### DETAILED DESCRIPTION OF THE PREFERRED EMBODIMENTS

The nail polish with a crack and color changing effect of the present invention includes a polymer, a solvent, a plasticizer, a pigment, a silica and a thermochromic powder.

The polymer accounts for 10-30 wt % (weight percent) of the total weight of the nail polish and may contain nitrocellulose and resin. The resin may be at least one selected from a group consisting of: phthalic anhydride, trimellitic anhydride, glycol copolymer and adipic acid, fumaric acid, phthalic acid, tricyclodecane dimethanol copolymer and a mixture thereof.

The solvent accounts for 44-64 wt % of the total weight of the nail polish and may contain butyl acetate, ethyl acetate and isopropyl alcohol.

The plasticizer accounts for 3-8 wt % of the total weight of the nail polish and may be acetyl tributyl citrate (ATBC).

The pigment accounts for 10-20 wt % of the total weight of the nail polish and may contain at least one selected from a group consisting of: red 6, red 7, yellow 5, red 34, mica, ferric ferrocyanide, titanium dioxide and black iron oxide. The color may be selected according to the requirement of color modulation.

The silica accounts for 8% of the total weight, which allows the nail polish of the present invention to generate a crack effect. After the silica contacts a nail base oil of the nail polish, the nail base oil of the nail polish generates a fast-dry and atomization phenomenon. Therefore, the original position of the silica becomes a blank area which forms cracks. (The steps of using the nail polish of the present invention generally include: (a) firstly applying a nail base oil of the nail polish on a nail; and (b) applying the nail polish of the present invention on the nail base oil of the nail polish.)

The thermochromic powder accounts for 5 wt % of the total weight, which allows the nail polish to change color at one or more specific temperatures. Moreover, the weight percent of the pigment and the thermochromic powder is 2:1.

The thermochromic powder is a material capable of providing color changing effect under heating. Based on different color changing manners, the thermochromic powder may be divided into reversible monocolor changing, irreversible monocolor changing, reversible multicolor changing, irreversible multicolor changing and the like. In brief, the thermochromic powder may change color with the changing of the temperature, and present different colors at different temperatures. Based on the category of the thermochromic powder having the thermal color changing characteristic, the thermochromic powder may be divided into the crystal structure changing type, thermal decomposition type, crystal transition type and the like. The color changing manner includes but not limited to: rose-red to colorless, purple-red to colorless, blue to colorless, green to colorless and the like.

In a preferred embodiment, the thermochromic powder may be composed of a plurality of micro capsules, and the micro capsules contain dyes and relevant polymers, and the dye may be spirolactones, fluorans, fulgides and the like.

In a preferred embodiment, the thermochromic powder may be in a colored state. When the temperature rises to 31° C., the thermochromic powder changes to be colorless. Therefore, after the thermochromic powder is blended with the pigment, the material of the thermochromic powder achieves the color changing effect with the changing of the temperature.

The components of the nail polish of the present invention in the preferred embodiments are listed as follows.

COMPONENTS OF THE NAIL POLISH OF THE
PRESENT INVENTION

| | Chemical | |
|---|---|---|
| | Abstracts Service | Weight |
| Name | (CAS No.) | Percentage |
| Butyl Acetate | 141-78-6 | 24.52%-29.5% |
| Ethyl Acetate | 123-86-4 | 14.94%-17.94% |
| Nitrocellulose | 9004-70-0 | 9.91%-12.91% |
| Acetyl Tributyl Citrate | 77-90-7 | 6.86% |
| Phthalic Anhydride/Trimellitic | | 9.19% |
| Anhydride/Glycol Copolymer | | |
| Isopropyl Alcohol | 67-63-0 | 5.91% |
| Stearalkonium Hectorite | 94891-33-5 | 1.34% |
| Adipic Acid/Fumaric | 24807-73-0 | 1.33% |
| Acid/Phthalic | | |
| Acid/Tricyclodecane | | |
| Dimethanol Copolymer | | |
| Citric Acid(for regulating the | 77-92-9 | 0.02% |
| PH value) | | |
| Pigment | | 10% |
| Thermochromic Powder | | 5% |
| Silica | | 8% |

The preparation of the nail polish of the present invention capable of generating cracks is carried out at the room temperature (25° C., where the preparation temperature ranges from 0° C. to 50° C.) and the preparation steps are described as follows.

1. The nail base oil is added in a blending barrel and meanwhile a blender is used to stir persistently in the blending barrel. The nail base oil is a mixture of Butyl Acetate, Ethyl Acetate, Nitrocellulose, Acetyl Tributyl Citrate, Isopropyl Alcohol and Stearalkonium Hectorite in the aforementioned table.

2. The silica is added in the blending barrel.

3. The butyl acetate is added in the blending barrel and is stirred persistently.

4. The pigment is added and then the thermochromic powder is added in the blending barrel and is stirred persistently.

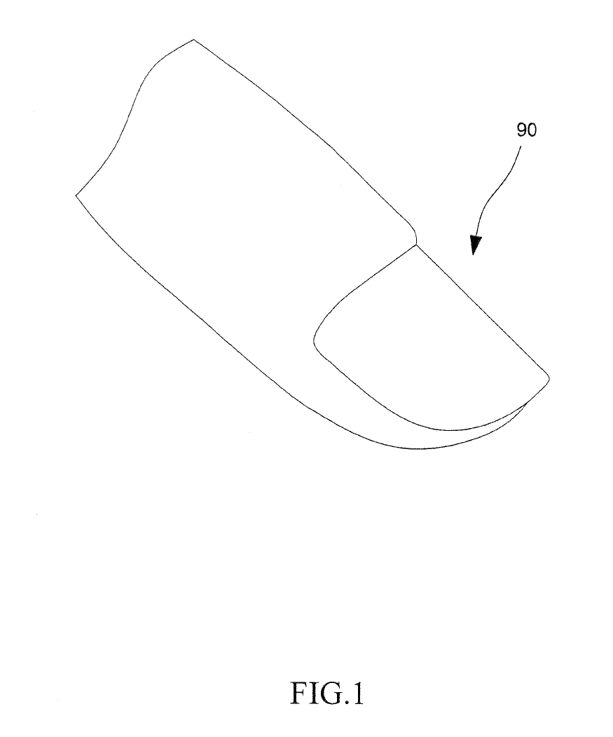

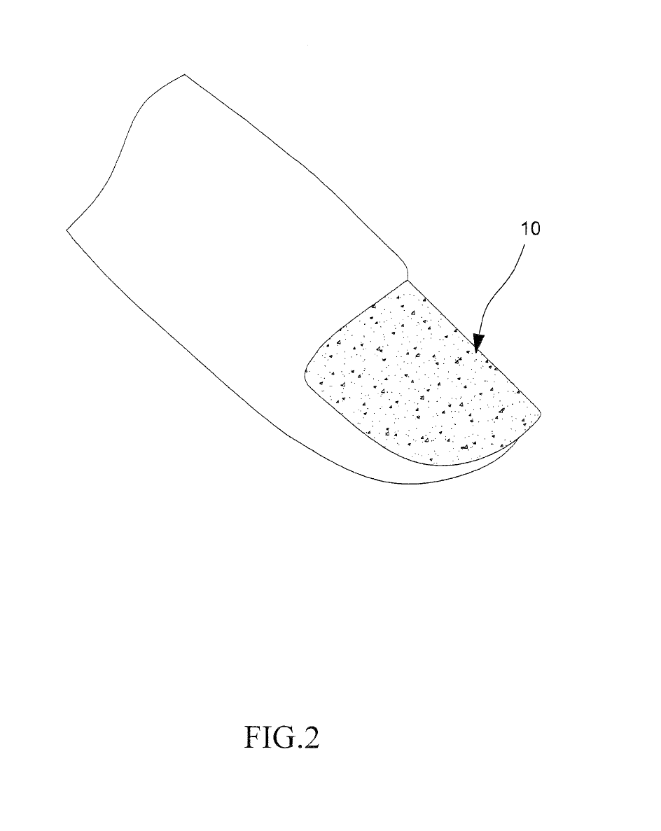

Referring to FIGS. 1 and 2, the nail polish (10) with a crack and color changing effect of the present invention is applied on a nail (90). The nail (90) is applied with the nail base oil of the nail polish before the nail polish of the present invention is applied, so the nail polish of the present invention is coated on the nail base oil of the nail polish.

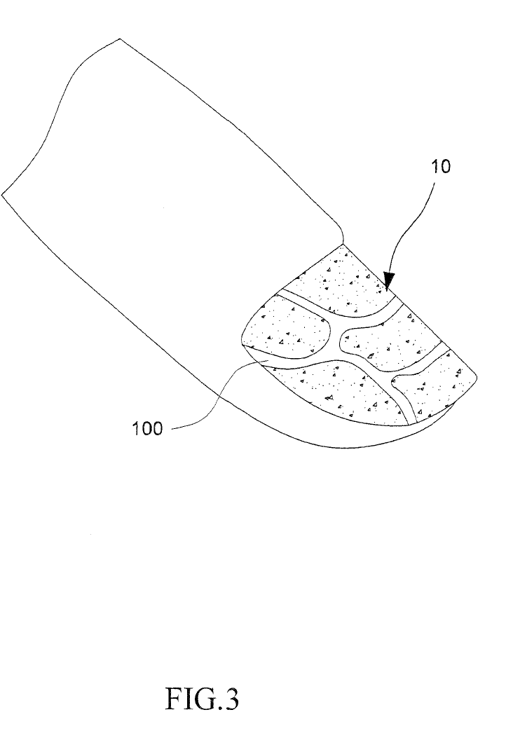

Further referring to FIG. 3, the nail polish (10) generates cracks (100) after the solvent is volatilized and the nail polish (10) is dried. The distribution of the cracks (100) differs according to different directions and manners of applying the nail polish (10).

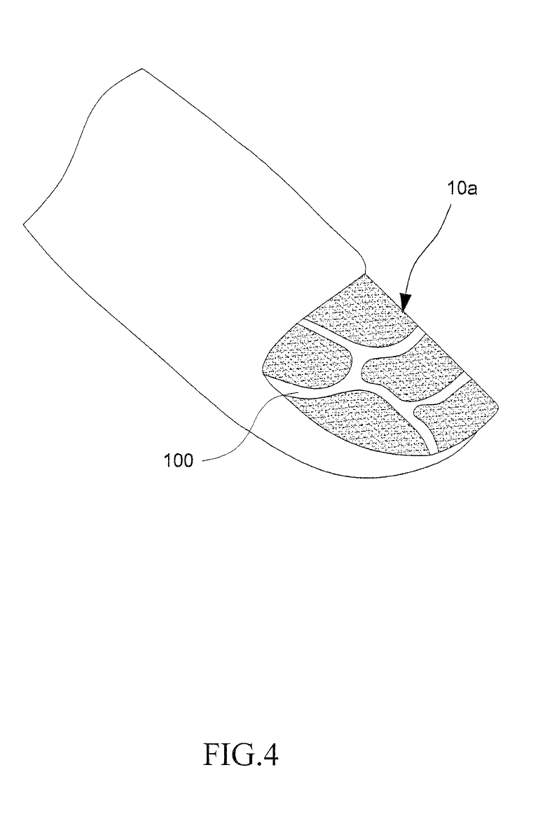

Further referring to FIG. 4, the dried nail polish (10) changes color when the nail temperature rises or drops to different specific temperatures, so the nail polish (10a) may present another visual effect after changing color. Moreover, since the temperature of the nail is gradually lowered from inside to outside, the color of the nail polish changes as the temperature changes, thus forming a gradient color distribution. Definitely, if the temperature of the nail is consistent, the formed color is identical. Moreover, the color changing process may be reversible and irreversible, and the color changes one time at a specific temperature or the color changes plural times at different specific temperatures, thus forming different variations without replacing the nail polish.

By the aforementioned technical means, the nail polish with a crack and color changing effect of the present invention may generate a variety of crack effects and further generate a color changing effect at a specific temperature, which greatly improves the applicability of the nail polish.

1. A nail polish with a crack and color changing effect, configured for applying on a nail which is already applied with a nail base oil thereon, the nail base oil being a mixture of butyl acetate, ethyl acetate, nitrocellulose, acetyl tributyl citrate, isopropyl alcohol and stearalkonium hectorite, the nail polish comprising:
a polymer, accounting for 10%-30% of the total weight and containing a nitrocellulose and a resin;
a solvent, accounting for 44%-64% of the total weight;
a plasticizer, accounting for 3%-8% of the total weight;
a pigment, accounting for 10%-20% of the total weight;
a silica, accounting for 8% of the total weight; and
a thermochromic powder, accounting for 5% of the total weight,
wherein after the nail polish is applied on the nail base oil applied on the nail, the silica contacts the nail base oil, and the nail base oil generates a fast-dry and atomization phenomenon, and the original position of the silica becomes a blank area which forms cracks.

2. The nail polish with a crack and color changing effect according to claim 1, wherein the plasticizer is acetyl tributyl citrate (ATBC).

3. The nail polish with a crack and color changing effect according to claim 1, wherein the nitrocellulose and resin respectively account for 8-10% and 90-92% of the total weight of the polymer.

4. The nail polish with a crack and color changing effect according to claim 1, wherein the thermochromic powder is in a colored state below a specific temperature and turns into a colorless state when the temperature rises to the specific temperature.

5. The nail polish with a crack and color changing effect according to claim 1, wherein the resin of the polymer is at least one selected from a group consisting of: phthalic anhydride, trimellitic anhydride, glycol copolymer and adipic acid, fumaric acid, phthalic acid, tricyclodecane dimethanol copolymer and a mixture thereof.

6. The nail polish with a crack and color changing effect according to claim 5, wherein the plasticizer is acetyl tributyl citrate (ATBC).

7. The nail polish with a crack and color changing effect according to claim 5, wherein the nitrocellulose and resin respectively account for 8-10% and 90-92% of the total weight of the polymer.

8. The nail polish with a crack and color changing effect according to claim 5, wherein the thermochromic powder is in a colored state below a specific temperature and turns into a colorless state when the temperature rises to the specific temperature.

9. The nail polish with a crack and color changing effect according to claim 1, wherein the solvent contains: butyl acetate, ethyl acetate and isopropyl alcohol.

10. The nail polish with a crack and color changing effect according to claim 9, wherein the plasticizer is acetyl tributyl citrate (ATBC).

11. The nail polish with a crack and color changing effect according to claim 9, wherein the nitrocellulose and resin respectively account for 8-10% and 90-92% of the total weight of the polymer.

12. The nail polish with a crack and color changing effect according to claim 9, wherein the thermochromic powder is in a colored state below a specific temperature and turns into a colorless state when the temperature rises to the specific temperature.
